(19) United States
(12) Patent Application Publication
(10) Pub. No.: US 2003/0028027 A1
(43) Pub. Date: Feb. 6, 2003

(54) Paroxetine maleate

(75) Inventors: Victor Witold Jacewicz, Tonbridge Wells (GB); David Alan Jones, Sevenoaks (GB); John Man, Sidcup (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(73) Assignee: SmithKline Beecham plc

(21) Appl. No.: 10/174,237

(22) Filed: Jun. 17, 2002

Related U.S. Application Data
(63) Continuation of application No. 09/647,792, filed on Nov. 22, 2000.
Application No. 09/647,792 is a 371 of international application No. PCT/GB99/01106, filed on Apr. 9, 1999.

(30) Foreign Application Priority Data
Apr. 9, 1998 (GB) 9807627.6
Oct. 30, 1998 (GB) 9823856.1

(51) Int. Cl. C 07D 4 7/02

(52) U.S. Cl. 546/197

(57) ABSTRACT

Paroxetine maleate and its preparation and use in the treatment and prevention of inter alia depression.

The present invention relates to a novel compound, to processes for preparing it and to its use in treating medical disorders.

Pharmaceutical products with antidepressant and anti-Parkinson properties are described in U.S. Pat. No. 3,912,743 and U.S. Pat. No. 4,007,196. An especially important compound among those disclosed is paroxetine, the (−)trans isomer of 4-(4′-fluorophenyl)-3-(3′,4′-methylenedioxy-phenoxymethyl)-piperidine. This compound is used in therapy as the hydrochloride salt for the treatment and prophylaxis of inter alia depression, obsessive compulsive disorder (OCD) and panic.

Example 2 of U.S. Pat. No. 4,007,196 describes the preparation of paroxetine by demethylation of the N-methyl derivative. Paroxetine free base is isolated as an oil by evaporation of a benzene solution. The free base is dissolved in ether and treated with a solution of maleic acid in ethyl ether to form a crystalline product, which is recrystallised from 99% ethanol-ether to give a maleate salt melting 136-8° C. Apart from the melting point, there is no characterizing data that allows an unambiguous assignment of structure.

Further, neither benzene nor ether are desirable as solvents for commercial manufacture, since high flammability, potentially explosive residues, and toxic residues would require expensive control measures. Also the process described in Example 2 of U.S. Pat. No. 4,007,196 is unsuitable for commercial use since it results in a heavy sticky gum.

We have now surprisingly discovered novel maleate salts of paroxetine which may be used as an alternative to the currently marketed hydrochloride, or as an intermediate in the preparation of the hydrochloride, and novel methods of preparation which are suitable for commercial use.

In one aspect of the present invention there is provided paroxetine maleate in which the ratio of paroxetine to maleic acid (by mole) is 1:1. In another aspect of the present invention there is provided paroxetine maleate in which the ratio of paroxetine to maleic acid (by mole) is 2:1.

In the 1:1 salt, the maleate anion may be associated with a proton (hydrogen atom) in addition to paroxetine or may be associated with another cation, for example an alkali metal or ammonium cation. In the former case the 1:1 salt may be described as paroxetine hydrogen maleate, while in the latter case the salt may be described as a mixed salt

In one aspect the novel salts of this invention are provided in non-crystalline form, which may a solid or an oil. The oil is preferably absorbed on a solid carrier, especially a carrier that is usable as a component of a pharmaceutical composition.

In another aspect the novel salts of this invention are provided in crystalline form. When the crystalline form exists as more than one polymorph, each polymorph forms another aspect of this invention.

We have discovered that crystalline paroxetine maleate in which the ratio of paroxetine to maleic acid (by mole) is 1:1 exists in at least two polymorphic forms.

Accordingly a further aspect of the invention provides paroxetine (1:1) maleate Form A having a melting point of 139-141° C. and having an IR or XRD spectrum substantially as disclosed in Example 1 below; and paroxetine (1:1) maleate Form B having a melting point of 136-138° C. and having an IR or XRD spectrum substantially as disclosed in Example 2 below.

The above-mentioned paroxetine maleates may be prepared by contacting appropriate stoichiometric amounts of the acid and paroxetine free base. Preferably the base is in solution, more preferably both are in solution. Mixed salts may be prepared by forming the precursor 1:1 salt in situ or using it pre-formed; and contacting it in solution with a solution containing the metal or ammonium ion.

Most commonly used solvents are suitable for mobilising paroxetine free base, for example toluene, alcohols such as methanol, ethanol, propan-2-ol, esters such as ethyl acetate, ketones such as acetone and butanone, halogenated hydrocarbons such as dichloromethane, and ethers such as tetrahydrofuran and diethyl ether. The maleic acid may be added as a solid, but is preferably added as a solution in an organic solvent such as ethanol or ethyl acetate, or water, methanol, propan-2-ol, or acetone. The maleic acid may also be added in the form of a soluble salt, for example ammonium maleate, or the maleic acid salt of an amine, for example ethylamine or diethylamine.

The concentration of paroxetine base is preferably in the range 5 to 50% weight/volume, more preferably in the range 10 to 30%. The concentration of maleic acid when used in solution is preferably in the range 0.1 to 5, preferably 0.5 to 2 molar. Elevated temperatures may be used to increase solubility.

The salt may be isolated in solid form by conventional means from a solution thereof obtained as above. For example, a non-crystalline salt may be prepared by precipitation from solution, spray drying and freeze drying of solutions, evaporating a solution to a glass, or vacuum drying of oils, or solidification of melts obtained from reaction of the free base and the acid.

A crystalline salt may be prepared by directly crystallising from a solvent in which the product has limited solubility, or by triturating or otherwise crystallising a non-crystalline salt. For example, paroxetine maleate may be recrystallised from a variety of organic solvents, such as acetonitrile, butanone, sec-butanol, dichloromethane, ethanol, 3-pentanone, propan-2-ol and toluene. An improved yield of the salt is obtained by evaporation of some or all of the solvent or by crystallisation at elevated temperature followed by controlled cooling, preferably in stages. Careful control of precipitation temperature and seeding may be used to improve the reproducibility of the production process and the particle size distribution and form of the product. Individual polymorphs are preferably crystallized directly from a solution of the salt, although recrystallizing a solution of one polymorph using seeds of another polymorph may also be carried out.

Thus paroxetine (1:1) maleate Form A may be prepared by crystallisation from a solution of paroxetine maleate, which may be prepared, for example, by mixing together a solution of paroxetine free base and a solution of maleic acid. Advantageously an excess of maleic is used, for example a molar ratio between 1:1 and 1:1.5, preferably between 1.1 and 1.3. Suitable solvents include ethyl acetate, methanol, ethanol, propan-2-ol, propan-1-ol, sec-butanol, butan-1-ol, methyl isobutylketone, acetone and acetonitrile, or a mixture of solvents, including mixtures with toluene.

The maleate Form B may be prepared by recrystallisation of Form A (or vice versa). More preferably, and advantageously, Form B is prepared directly by crystallisation in a similar manner to Form A, that is directly from a solution of paroxetine maleate, which may be prepared by mixing together a solution of paroxetine free base, typically as the final stage of a manufacturing process, and a solution of maleic acid. A wide range of solvents may be used, particularly if seeding is also used to ensure formation of the desired polymorph, but preferred solvents include toluene, butanone, acetone and dichloromethane. Propan-2-ol may also be used.

Seeding with the desired polymorph may be incorporated into the process to ensure reliability of polymorph formation and to control crystal size distribution.

It has been found that toluene or mixtures containing toluene are among the most suitable solvents for the preparation of paroxetine maleate Form B.

The processing properties of Form B are generally superior to those of Form A, since this polymorph is more granular and easier to filter, wash and dry. Consequently, this invention provides a particularly convenient manufacturing process for paroxetine (1:1) maleate, in which unsuitable solvents are avoided and a solution of paroxetine free base is prepared in toluene and converted directly to the maleate salt. Known processes for the preparation of paroxetine utilize toluene as the solvent of choice. Before the invention of paroxetine maleate Form B disclosed herein, there was no process available which had the convenience of using toluene solutions of free base as starting material, since treatment of such solutions generated paroxetine maleate in the form of an oil or gum.

An alternative method of preparing paroxetine maleate is to start with a salt of paroxetine with an organic acid, such as acetic acid, rather than using paroxetine free base. Use of another salt of paroxetine as a starting material is suitable for preparation of the crystalline salt or, if a volatile acid such as acetic acid is used, non-crystalline salts by methods that involve evaporation (such as freeze-drying and spray-drying).

The salt may obtained as a solvate, when during isolation from solution it becomes associated with the solvent in which it is dissolved. Any such solvate forms a further aspect of this invention. Solvates may be returned to the unsolvated salt by heating, for example by oven-drying, or by treatment with a displacement solvent which does not form a solvate.

Prior to the isolation of the paroxetine maleate, water may be removed from the solution containing the salt by azeotropic distillation to avoid the formation of hydrates or to obtain the product in anhydrous form. In that case, suitable solvents for the solution of the salt are those which form an azeotrope with water such as toluene and propan-2-ol. It should also be appreciated that mixtures of solvents can also be used to aid the azeotropic removal of water.

Paroxetine free base may be prepared according to the procedures generally outlined in U.S. Pat. No 4,007,196 and EP-B-0 223403. Maleic acid is commercially available.

The compounds of this invention may be used to treat and prevent the following disorders:

| | |
|---|---|
| Alcoholism | Anxiety |
| Depression | Obsessive Compulsive Disorder |
| Panic Disorder | Chronic Pain |
| Obesity | Senile Dementia |
| Migraine | Bulimia |
| Anorexia | Social Phobia |
| Pre-Menstrual Syndrome (PMS) | Adolescent Depression |
| Trichotillomania | Dysthymia |
| Substance Abuse | |

These disorders are herein after referred to as “the Disorders”.

The present invention further provides a method for treating and/or preventing any one or more of the Disorders by administering an effective and/or prophylactic amount of a salt of the invention to a sufferer in need thereof.

The present invention further provides a pharmaceutical composition for use in the treatment and/or prevention of the Disorders which comprises an admixture of a salt of the invention with a pharmaceutically acceptable carrier.

The present invention also provides the use of a salt of the invention for treating and/or preventing the Disorders.

The present invention also provides the use of a salt of the invention in the manufacture of a medicament for treating and/or preventing the Disorders.

Most suitably the present invention is applied to the treatment of depression, OCD and panic.

Compositions containing the salt of this invention may be formulated for administration by any route, and examples are oral, sub-lingual, rectal, topical, parenteral, intravenous or intramuscular administration. Preparations may, if desired, be designed to give slow release of the paroxetine salt.

The medicaments may, for example, be in the form of tablets, capsules, sachets, vials, powders, granules, lozenges, reconstitutable powders, or liquid preparations, for example solutions or suspensions, or suppositories.

The composition is usually presented as a unit dose composition containing from 1 to 200 mg of active ingredient calculated on a free base basis, more usually from 5 to 100 mg, for example 10 to 50 mg such as 10, 12.5, 15, 20, 25, 30 or 40 mg by a human patient. Most preferably unit doses contain 20 mg of active ingredient calculated on a free base basis. Such a composition is normally taken from 1 to 6 times daily, for example 2, 3 or 4 times daily so that the total amount of active agent administered is within the range 5 to 400 mg of active ingredient calculated on a free base basis. Most preferably the unit dose is taken once a day.

Preferred unit dosage forms include tablets or capsules.

The compositions of this invention may be formulated by conventional methods of admixture such as blending, filling and compressing.

Suitable carriers for use in this invention include a diluent, a binder, a disintegrant, a colouring agent, a flavouring agent and/or preservative. These agents may be utilized in conventional manner, for example in a manner similar to that already used for marketed anti-depressant agents.

Specific examples of pharmaceutical compositions include those described EP-B-0-223403, and U.S. Pat. No. 4,007,196 in which the products of the present invention may be used as the active ingredients.

The following Examples illustrate the present invention:

### EXAMPLE 1

### Preparation of Paroxetine (1:1) Maleate Form A

Maleic acid [14.8 g,0.128 mol] was stirred in ethyl acetate [100 ml] and the solution warmed gently. Paroxetine free base [42.7 g] in ethyl acetate was then added rapidly with stirring and the suspension briefly became clear then immediately solidified. Warming was continued until the solution was at reflux and the mixture was then stirrable. The reaction mixture was allowed to cool with stirring and the cold solution filtered and washed with ethyl acetate [25 ml ] and dried in a vacuum oven at 40 C. for 3 hours to give paroxetine maleate Form A. NMR showed a ratio of 1:1 for paroxetine: maleic acid.

m.pt. 139-141° C. IR 1674, 1360, 1306, 1269, 1248, 1190, 1137, 1101, 1026, 863, 834, 783, 706, 597.

XRD

| Angle | d-value | Peak Int. |
|---|---|---|
| [° 2 q] | a [Å] | counts |
| 5.10 | 17.30 | 8649 |
| 8.04 | 10.99 | 106 |
| 8.78 | 10.10 | 64 |
| 10.26 | 8.62 | 692 |
| 11.98 | 7.38 | 77 |
| 13.91 | 6.36 | 829 |
| 15.43 | 5.74 | 15500 |
| 15.88 | 5.58 | 5242 |
| 16.13 | 5.49 | 586 |
| 17.71 | 5.00 | 745 |
| 18.67 | 4.75 | 635 |
| 19.37 | 4.59 | 475 |
| 20.29 | 4.37 | 1544 |
| 20.52 | 4.32 | 1945 |
| 20.90 | 4.25 | 493 |
| 21.32 | 4.16 | 384 |
| 23.18 | 3.83 | 376 |
| 24.30 | 3.66 | 2663 |
| 25.00 | 3.56 | 912 |
| 25.80 | 3.45 | 566 |
| 26.52 | 3.36 | 1640 |
| 27.96 | 3.19 | 1616 |
| 28.76 | 3.10 | 671 |
| 30.00 | 2.98 | 1030 |
| 30.34 | 2.94 | 552 |
| 31.10 | 2.87 | 1980 |
| 32.10 | 2.79 | 1005 |
| 32.77 | 2.74 | 342 |
| 33.36 | 2.69 | 529 |

### EXAMPLE 2

### Paroxetine (1:1) Maleate Form B by recrystallization of Form A from butanone

A suspension of paroxetine maleate Form A (0.5 g) in butanone (4 ml) was stirred vigorously and heated to reflux. The solution was allowed to cool slowly to room temperature to give a paroxetine maleate Form B as a granular white crystalline solid which was collected by filtration and dried in vacuo over phosphorous pentoxide. NMR showed a ratio of 1:1 for paroxetine : maleic acid, butanone content was approximately 0.1% by weight.

m.pt. 136-138° C. IR(nujol mull), 1709, 1385, 1280, 1247, 1195, 1134, 1045, 931, 904, 876, 862, 838, 816, 805, 779, 714, 595, 543.

XRD

| Angle | d-value | Peak Int. |
|---|---|---|
| [° 2 q] | a [Å] | counts |
| 5.11 | 17.28 | 79 |
| 8.35 | 10.58 | 19 |
| 10.47 | 8.44 | 292 |
| 12.90 | 6.86 | 420 |
| 13.64 | 6.49 | 339 |
| 13.97 | 6.34 | 196 |
| 14.41 | 6.14 | 428 |
| 15.51 | 5.71 | 724 |
| 16.32 | 5.43 | 1624 |
| 16.60 | 5.34 | 973 |
| 17.78 | 4.98 | 1332 |
| 19.75 | 4.49 | 3102 |
| 20.21 | 4.39 | 5868 |
| 20.68 | 4.29 | 729 |
| 21.25 | 4.18 | 420 |
| 21.95 | 4.04 | 1197 |
| 22.13 | 4.01 | 876 |
| 22.80 | 3.89 | 581 |
| 23.65 | 3.76 | 576 |
| 24.46 | 3.64 | 1747 |
| 24.97 | 3.56 | 5227 |
| 25.88 | 3.44 | 955 |
| 27.69 | 3.22 | 454 |
| 28.23 | 3.16 | 566 |
| 29.26 | 3.05 | 2470 |
| 29.94 | 2.98 | 1866 |
| 30.76 | 2.90 | 552 |
| 31.26 | 2.86 | 697 |
| 32.02 | 2.79 | 605 |
| 32.44 | 2.75 | 557 |
| 33.01 | 2.71 | 571 |
| 33.88 | 2.64 | 458 |

### EXAMPLE 3

### Paroxetine (1:1) Maleate Form A by recrystallization from propan-2-ol

A suspension of paroxetine maleate Form A (0.5 g) in propan-2-ol (4 ml) was stirred vigorously and heated to reflux. The solution was allowed to cool to room temperature to give a paroxetine maleate Form A as white plate-like crystals which were collected by filtration and dried in vacuo over phosphorous pentoxide. The infra-red spectrum was the same as for the product of Example 1.

### EXAMPLE 4

### Paroxetine (1:1) Maleate Form A by recrystallization from acetonitrile

A suspension of paroxetine maleate Form A (0.5 g) in acetonitrile (4 ml) was stirred vigorously and heated to reflux to dissolve. The solution was allowed to coot to room temperature to give paroxetine maleate Form A as large plate-like crystals. The crystalline solid was isolated by filtration and dried in vacuo over phosphorous pentoxide. The infra-red spectrum was the same as for the product of Example 1.

### EXAMPLE 5

### Paroxetine (1:1) Maleate Form B by recrystallization of Form A from toluene

A suspension of paroxetine maleate Form A (0.5 g) in toluene (8 ml) was stirred vigorously with heating and approximately half the solvent was distilled off. A further 3 ml toluene was added and the reaction mixture cooled to room temperature. After 2 hours, the white solid which had formed was collected by filtration and dried, to afford paroxetine maleate Form B as a granular solid. The infra-red spectrum was the same as for the product of Example 2.

### EXAMPLE 6

### Paroxetine (1:1) Maleate Form A by recrystallization from sec-butanol

A suspension of paroxetine maleate Form A (0.5 g) in sec-butanol (4 ml) was stirred vigorously and heated to reflux. The solution was allowed to cool to room temperature to produce paroxetine maleate Form A as a white crystalline solid, which was collected by filtration and dried in vacuo over phosphorous pentoxide. The infra-red spectrum was the same as for the product of Example 1.

### EXAMPLE 7

### Paroxetine Maleate Form B by recrystallization of Form A from dichloromethane

A suspension of paroxetine maleate Form A (0.5 g) in dichloromethane (3 ml) was stirred vigorously and heated to reflux. The solution was allowed to cool to room temperature for 5 hours to produce paroxetine maleate form B as a white granular solid. The product was collected by filtration and dried in vacuo over phosphorous pentoxide.

The infra-red spectrum was the same as for the product of Example 2.

### EXAMPLE 8

### Paroxetine (1:1) Maleate Form A by recrystallization from ethanol

A suspension of paroxetine maleate Form A (0.5 g) in ethanol (3 ml) was stirred vigorously and heated to reflux. The solution was allowed to cool to room temperature to give paroxetine maleate Form A as a white crystalline solid which was collected by filtration and dried in vacuo over phosphorous pentoxide. The infra-red spectrum was the same as for the product of Example 1.

### EXAMPLE 9

### Paroxetine (1:1) Maleate Form B by recrystallization of Form A from 3-pentanone

A suspension of paroxetine maleate Form A (0.5 g) in 3-pentanone (4 ml) was stirred vigorously and heated to reflux. The solution was allowed to cool to room temperature to give a paroxetine maleate Form B as a crystalline solid. The product was collected by filtration and dried in vacuo over phosphorous pentoxide. The infra-red spectrum was the same as for the product of Example 2.

### EXAMPLE 10

### Preparation of Paroxetine (1:1) Maleate Form A

To a solution of paroxetine free base (15.16 g) in toluene (75 ml) was added a solution of maleic acid (5.34 g) in ethyl acetate (37.5 ml) with vigorous stirring A solid product crystallized out quickly and after 20 minutes was collected by filtration and dried in vacuo at 40° C. The infra-red spectrum was the same as for the product of Example 1.

Yield: [18.60 g, 41 mmol, 90.7%]

### EXAMPLE 11

### Preparation of Paroxetine (1:1) Maleate Form B

N-phenyloxycarbonyl paroxetine (5.00 kg ), potassium hydroxide flake (4.50 kg) and toluene (75.0 liters) was heated to reflux under a nitrogen atmosphere with good stirring over a period of 40 minutes. After stirring for 4 hr 45 min at reflux the contents of the reactor was allowed to cool to room temperature. Water (50 liters) was added and the mixture stirred for 30 minutes and then allowed to settle for 30 minutes. The lower aqueous layer was drained from the reactor and the remaining toluene solution was heated to reflux. Water was removed by heating to reflux with Dean and Stark apparatus. Toluene (11.5 liters) was added and a similar quantity of the reaction solvent removed by distillation. The remaining mixture was cooled to approximately 100° C., stirred vigorously, and a mixture of maleic acid (1.04 kg) and paroxetine maleate Form B (40 g) seed crystals added in four portions via a solids charging hopper. The temperature was reduced in stages whereupon crystallization commenced between 60° C. and 50° C. The temperature was held at 40° C. for two hours for the bulk of the crystallization to occur. Finally the temperature was reduced to approximately 15° C. and the product Form B paroxetine maleate collected in a centrifuge, washed with toluene and dried in a vacuum oven at 50° C. Yield 3.1 kg.

### EXAMPLE 12

### Preparation of Paroxetine (2:1) Maleate

A solution of maleic acid [0.74 g] in ethanol [100 ml] was added to a solution of paroxetine in toluene [10 ml of a 0.42 g/ml solution] and the solution stirred for 30 minutes at ambient temperature. The solvent was removed by evaporation in vacuo to give a viscous oil, which was redissolved in chloroform. The chloroform solution was evaporated to dryness to give a white solid.

NMR indicated a molar ratio 2:1 paroxetine to maleic acid.

### EXAMPLE 13

| INGREDIENTS | 20 mg Tablet | 30 mg Tablet |
|---|---|---|
| Paroxetine maleate (1:1 or 2:1) | 20.00 mg based | 30.0 mg based |
| | on free base | on free base |
| Dicalcium Phosphate (DCP) | 83.34 mg | 125.0 mg |
| Microcrystalline Cellulose | 50.67 mg | 76.0 mg |
| Sodium Starch Glycollate | 8.34 mg | 12.5 mg |
| Magnesium Stearate | 1.67 mg | 2.5 mg |

Commercial source of the ingredients

| | |
|---|---|
| Dicalcium Phosphate Dihydrate | Emcompress or Ditab* |
| Microcrystalline Cellulose | Avicel PH 102* |
| Sodium Starch Glycollate | Explotab.* |

*Tradenames

### Method

1. Pass DCP through a screen and weigh it into a Planetary mixer.

2. Add 30 mesh Paroxetine to the bowl.

3. Add 20 mesh Avicel and Explotab and mix all the powders for 10 minutes.

4. Add magnesium stearate and mix for 5 minutes.

### Tablet into Pentagonal Tablets Using the Following Punches

| | | |
|---|---|---|
| 30 mg Tablet | 9.5 mm | Circumcircle |
| 20 mg Tablet | 8.25 mm | Circumcircle |

The tablets are made satisfactorily on a single punch or a Rotary press.

### EXAMPLE 14

| INGREDIENTS | 10 mg Tablet | 20 mg Tablet | 30 mg Tablet |
|---|---|---|---|
| Paroxetine maleate | 10 mg | 20 mg | 30 mg based |
| (1:1 or 2:1) | based on | based on | on free base |
| | free base | free base | |
| Sodium Starch Glycollate | 2.98 mg | 5.95 mg | 8.93 mg |
| Granular Dicalcium | 158.88 mg | 317.75 mg | 476.63 mg |
| Phosphate | | | |
| (DITAB) or Dicafos | | | |
| Magnesium Stearate | 1.75 mg | 3.50 mg | 5.25 mg |

### Method

1. Paroxetine, Sodium Starch Glycollate and Dicalcium Phosphate Dihydrate are screened and mixed together in a suitable mixer. (Planetary, Cuble or High Energy Shear mixer.)

2. Add Magnesium Stearate and compress it into a tablet using a single punch or Rotary Tablet machine.

1. Paroxetine maleate in which the ratio of paroxetine to maleic acid (by mole) is 1:1.

2. Paroxetine maleate in which the ratio of paroxetine to maleic acid (by mole) is 2:1.

3. A compound according to claim 1 or 2 in non-crystalline form.

4. A compound according to claim 1 or 2 in crystalline form.

5. Paroxetine (1:1) maleate Form A having a melting point of 139-141° C. and having an IR or XRD spectrum substantially as disclosed in Example 1.

6. Paroxetine (1:1) maleate Form B having a melting point of 136-138° C. and having an IR or XRD spectrum substantially as disclosed in Example 2.

7. A process for the preparation of a compound as claimed in claim 1, 2 or 3 by precipitation from a solution of a paroxetine maleate, spray drying or freeze drying a solution of a paroxetine maleate, evaporating a solution of a paroxetine maleate to a glass, or by vacuum drying of oils of a paroxetine maleate, or solidification of melts of a paroxetine maleate.

8. A process for the preparation of a compound as claimed in claim 1, 2, 4, 5 or 6 by crystallization or recrystallization from a solution of a paroxetine maleate.

9. A process for preparing paroxetine (1:1) maleate Form A by crystallisation from a solution of paroxetine maleate in ethyl acetate, methanol, ethanol, propan-2-ol, propan-1-ol, sec-butanol, butan-1-ol, methyl isobutylketone, acetone or acetonitrile, or a mixture of solvents, including mixtures with toluene.

10. A process for preparing paroxetine (1:1) maleate Form B by crystallisation from a solution of paroxetine maleate in toluene, or butanone, acetone, dichloromethane or propan-2-ol, or a mixture of one of said solvents with toluene.

11. A process according to claim 9 or 10 in which the solution is seeded with seed crystals of the desired polymorph.

12. A process according to claim 7, 8, 9, 10 or 11 in which the solution, oil or melt of a paroxetine maleate is prepared by treating paroxetine free base or an organic acid salt thereof with maleic acid or an ammonium or amine salt thereof.

13. A process for preparing paroxetine (1:1) maleate Form B in which a solution of paroxetine free base, which is the final stage of a process for manufacturing paroxetine, is treated with maleic acid, and Form B is crystallised from said solution.

14. A process according to claim 13, in which the paroxetine free base, or the resultant maleate is in solution in toluene, or in a solvent mixture containing toluene,

15. A method for treating and/or preventing any one or more of the Disorders by administering an effective and/or prophylactic amount of a salt of the invention to a sufferer in need thereof.
